(19) United States
(12) Patent Application Publication
(10) Pub. No.: US 2015/0025285 A1
(43) Pub. Date: Jan. 22, 2015

(54) REGENERATION OF OLEFIN TREATING ADSORBENTS FOR REMOVAL OF OXYGENATE CONTAMINANTS

(71) Applicants: Robert Fletcher Cleverdon, Walnut Creek, CA (US); Clifford Michael Lowe, Moraga, CA (US); Hye Kyung Cho Timken, Albany, CA (US)

(72) Inventors: Robert Fletcher Cleverdon, Walnut Creek, CA (US); Clifford Michael Lowe, Moraga, CA (US); Hye Kyung Cho Timken, Albany, CA (US)

(73) Assignee: Chevron U.S.A. Inc., San Ramon, CA (US)

(21) Appl. No.: 13/944,560

(22) Filed: Jul. 17, 2013

(51) Int. Cl. C07C 7/13; C07C 2/58

(52) U.S. Cl. CPC C07C 7/13; C07C 2/58
USPC 585/331; 585/733

(57) ABSTRACT

Processes for eliminating oxygenates and water from a light hydrocarbon processing system, wherein oxygenates are removed from a light hydrocarbon stream by adsorption of the oxygenates on an oxygenate adsorption unit to provide a deoxygenated hydrocarbon stream, the oxygenate adsorption unit is regenerated via a regenerant stream to provide an oxygenated regenerant stream comprising the oxygenates, and the oxygenated regenerant stream is subjected to hydro-deoxygenation to convert the oxygenates into paraffins and water, wherein the water may also be permanently removed from the system.

### TECHNICAL FIELD

The present invention relates to processes for regenerating olefin treating adsorbents for the removal of oxygenate contaminants.

### BACKGROUND

Various refinery and petrochemical processes involve reacting light olefins, to produce transportation fuels, plastics, and other commercial products, using catalyst systems that can be poisoned by contaminants in the olefin feed. Such contaminants may include water as well as various oxygenates, e.g., alcohols, ketones, carboxylic acids, and ethers.

Adsorbent materials for removing the water and oxygenates from the olefin feed become spent after use for a limited time period and must be regenerated for re-use to avoid excessive consumption and cost of the adsorbents. Spent adsorbent can be regenerated by desorbing the water and oxygenates into a stream of hot hydrocarbon vapor, e.g., isobutane. Such hydrocarbons may be valuable as feeds to various refinery processes. For example, isobutane is a valuable feed to ionic liquid alkylation. However, isobutane regenerant becomes contaminated with oxygenates and water during adsorbent regeneration. It is advantageous to remove the contaminants from the isobutane to prevent the accumulation of water and oxygenates, which could otherwise eventually break through the adsorbent beds and cause catalyst deactivation.

There is a need for processes for the elimination of oxygenate contaminants from light hydrocarbon processing systems in order to prevent contaminant accumulation in such systems, thereby protecting catalysts from deactivation by the contaminants.

### SUMMARY

In one embodiment there is provided a process for eliminating oxygenates from a light hydrocarbon processing system, the process comprising feeding an olefin stream to an oxygenate adsorption unit to provide a deoxygenated olefin stream; after the feeding step, desorbing oxygenates from the oxygenate adsorption unit via a regenerant stream to provide an oxygenated regenerant stream comprising the oxygenates; and converting the oxygenates of the oxygenated regenerant stream to paraffins and water.

In another embodiment there is provided a process for eliminating oxygenates from a light hydrocarbon processing system, the process comprising removing oxygenates from an olefin stream via an oxygenate adsorption unit to provide a deoxygenated olefin stream, wherein the oxygenate adsorption unit becomes spent; regenerating the spent oxygenate adsorption unit via a regenerant stream to provide an oxygenated regenerant stream comprising the oxygenates; and contacting the oxygenated regenerant stream with a hydro-deoxygenation catalyst in the presence of hydrogen gas in a hydro-deoxygenation zone under hydro-deoxygenation conditions, wherein the oxygenates of the oxygenated regenerant stream are converted to paraffins and water.

In a further embodiment there is provided a process for eliminating oxygenates from a light hydrocarbon processing system, the process comprising feeding an olefin stream to an oxygenate adsorption unit to provide a deoxygenated olefin stream; contacting the deoxygenated olefin stream and an isoparaffin stream with an ionic liquid catalyst in an ionic liquid alkylation zone under ionic liquid alkylation conditions; separating an alkylation hydrocarbon phase from an effluent of the ionic liquid alkylation zone; fractionating the alkylation hydrocarbon phase to provide an alkylate product; when the oxygenate adsorption unit becomes spent, regenerating the spent oxygenate adsorption unit via a regenerant stream to provide an oxygenated regenerant stream comprising oxygenates; and converting the oxygenates of the oxygenated regenerant stream to paraffins and water.

As used herein, the terms “comprising” and “comprises” mean the inclusion of named elements or steps that are identified following those terms, but not necessarily excluding other unnamed elements or steps.

### BRIEF DESCRIPTION OF THE DRAWINGS

FIG. 1 schematically represents a system and process for the elimination of oxygenates from hydrocarbon processing systems, according to an embodiment of the present invention;

FIG. 2 schematically represents the treatment of an oxygenate adsorption unit for the removal of residual olefins therefrom, according to another embodiment of the present invention; and

FIG. 3 schematically represents a system and process for ionic liquid catalyzed alkylation using a deoxygenated olefin stream, according to another embodiment of the present invention.

### DETAILED DESCRIPTION

Various refinery and petrochemical processes use light olefins, such as propene and butenes, as feeds to produce commercial products. An exemplary process is the alkylation of olefins with isobutane to produce high octane motor gasoline using ionic liquid catalysts. Refinery olefin streams, e.g., from a fluid catalytic cracking (FCC) unit, are typically contaminated with both water and oxygenates. It may be desirable or necessary to decrease the amount of water and/or oxygenates in olefin feeds for ionic liquid alkylation to very low levels before the olefin feed contacts the ionic liquid catalyst.

Adsorbent materials used for removing water and oxygenates from an olefin feed become spent after use for a limited time period. Spent adsorbent can be regenerated by desorbing the water and oxygenates into a regenerant stream, e.g., comprising hot hydrocarbon vapor. Oxygenates, such as alcohols and ketones, are typically more difficult to remove than water due to their much higher solubility in hydrocarbon liquids.

As disclosed herein, oxygenates as well as water can be permanently removed or eliminated from a light hydrocarbon processing system to prevent contaminant induced catalyst deactivation. For example, applicants have found that oxygenates can be removed from an oxygenated regenerant stream from an oxygenate adsorption unit by converting the oxygenates in the oxygenated regenerant stream to paraffins and water.

The term “deoxygenated” may be used herein to refer to a hydrocarbon stream from which one or more oxygenates may have been adsorbed or otherwise removed, such that the hydrocarbon feed stream or regenerant stream may be depleted in the one or more oxygenates; a deoxygenated stream may similarly be depleted in water.

The term “oxygenated” may be used herein to refer to a regenerant stream into which one or more oxygenates may have been desorbed, such that the regenerant stream may be enriched in the one or more oxygenates; an oxygenated stream may similarly be enriched in water.

Applicants have found that oxygenate and water may be effectively eliminated from olefin streams to provide deoxygenated olefin streams. Such olefin streams may be suitable for light hydrocarbon processing, including ionic liquid catalyzed alkylation.

Oxygenate Removal for Light Hydrocarbon Processing

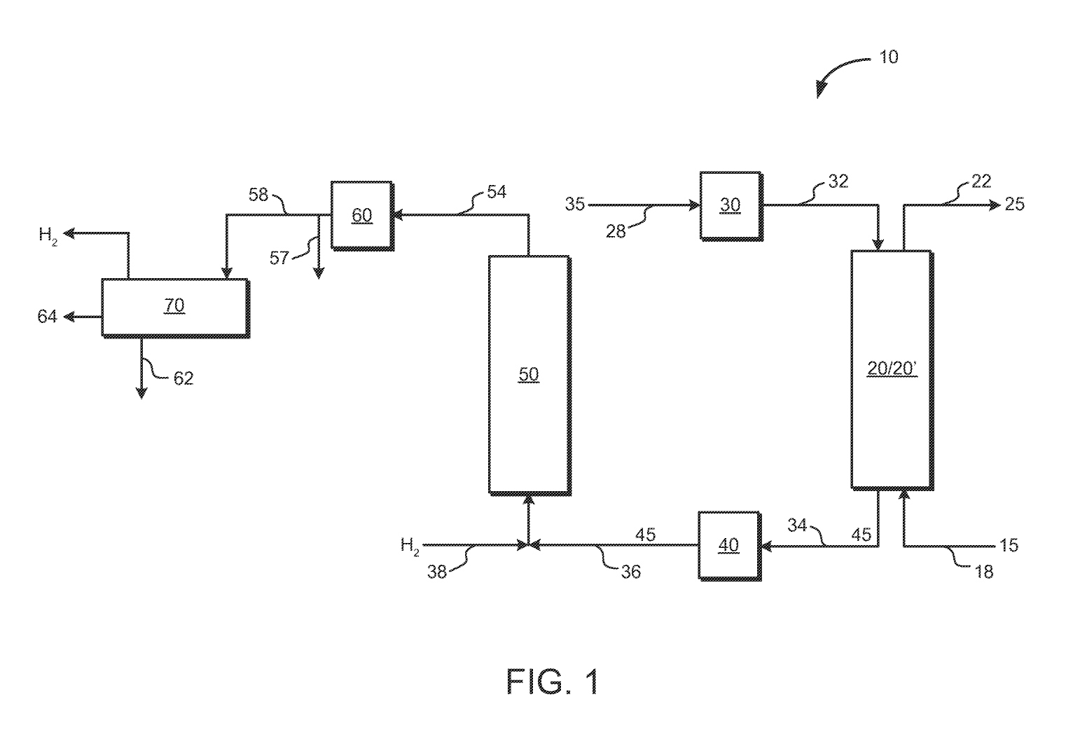

FIG. 1 schematically represents a process for the elimination of oxygenates from hydrocarbon processing systems, according to an embodiment of the present invention. System 10 may comprise an oxygenate adsorption unit 20/20′ that can be operated in an adsorption mode or a regeneration mode, 20, 20′, respectively. In the adsorption mode, an olefin stream 15 may be fed to oxygenate adsorption unit 20 via line 18. As an example, olefin stream 15 may comprise light olefins, such as C₃-C₅ olefins. Olefin stream 15 may be a raw or untreated olefin stream and may comprise water and/or oxygenate contaminants.

Oxygenate adsorption unit 20 may comprise an adsorbent for selectively adsorbing water and oxygenates from olefin stream 15. As a non-limiting example, an adsorbent of oxygenate adsorption unit 20 may comprise at least one of a molecular sieve and a metal oxide. Non-limiting examples of adsorbents for use in oxygenate adsorption unit 20 include a molecular sieve selected from the group consisting of silicates, aluminosilicates, aluminophosphates, silicoaluminophosphates, and combinations thereof. In a sub-embodiment, an adsorbent for use in oxygenate adsorption unit 20 may comprise a zeolite, such as zeolite 13×. The adsorbent of oxygenate adsorption unit 20 may be disposed in at least one adsorbent bed (not shown).

Oxygenate adsorption unit 20/20′ may be operated in the adsorption mode or the regeneration mode. The regeneration mode may also be referred to herein as a desorption mode. FIG. 1 shows the operation of oxygenate adsorption unit 20/20′ in the adsorption mode and in the regeneration mode, it being understood that oxygenate adsorption unit 20/20′ may be operated alternately in the adsorption and regeneration modes.

During the adsorption mode of oxygenate adsorption unit 20, water and oxygenate contaminants may be adsorbed from olefin stream 15. In an embodiment, during the adsorption mode, more than one oxygenate adsorption unit may be arranged in series for the adsorption of water and oxygenates from olefin stream 15. During the adsorption mode, oxygenate adsorption unit 20 may be maintained at a temperature typically in the range from 50 to 150° F. (10 to 65.56 degree Celsius), or from 70 to 130° F. (21.11 to 54.44 degree Celsius). The feed of olefin stream 15 to oxygenate adsorption unit 20 may be either upflow or downflow.

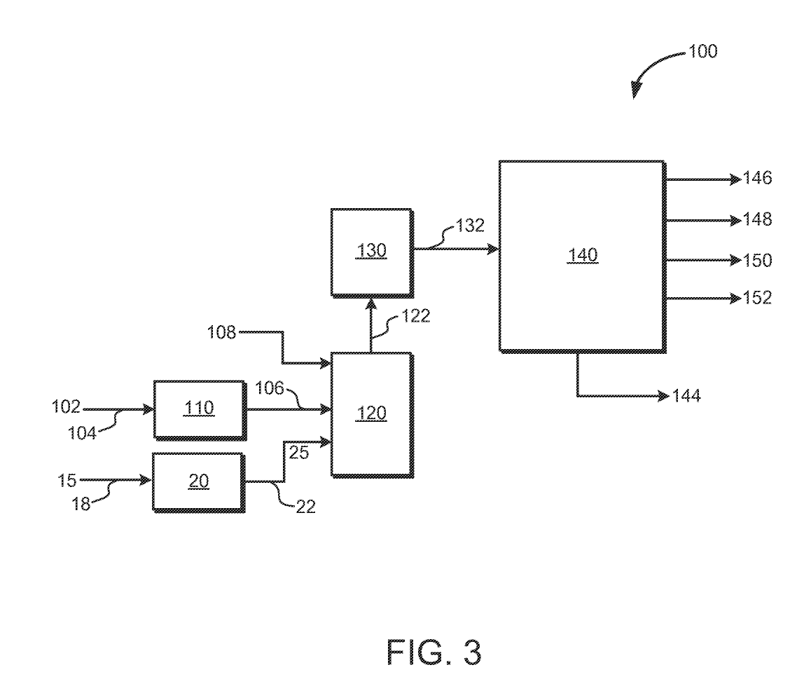

During the adsorption mode, a deoxygenated olefin stream 25 may be obtained from oxygenate adsorption unit 20. The expression “deoxygenated olefin stream” may be used herein to refer to an olefin stream that is depleted in oxygenates as compared with an untreated olefin stream. A deoxygenated olefin stream 25 (e.g., FIGS. 1 and 3) may also be depleted in water as compared with an untreated olefin stream, it being understood that water may be removed from an untreated olefin stream concurrently with oxygenate removal, e.g., by passage of the olefin stream 15 through oxygenate adsorption unit 20.

In an embodiment, deoxygenated olefin stream 25 may have an oxygenate content of not more than 5 ppmw, or not more than 2 ppmw, or not more than 1 ppmw. In an embodiment, deoxygenated olefin stream 25 may have a water content of not more than 5 ppmw, or not more than 2 ppmw, or not more than 1 ppmw. Deoxygenated olefin stream 25 may be fed via line 22 to one or more downstream unit operations. In an embodiment, deoxygenated olefin stream 25 may be fed to an ionic liquid alkylation zone 120 (see, for example, FIG. 3).

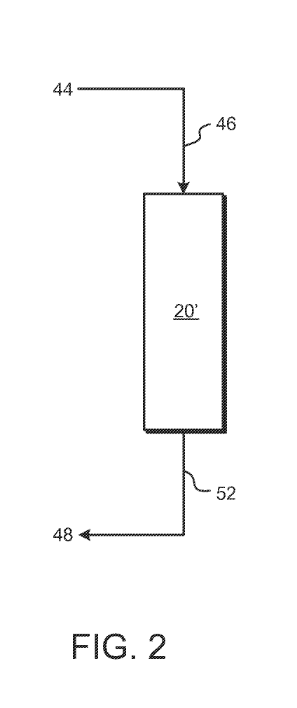

Although only one oxygenate adsorption unit 20/20′ is shown in FIG. 1, a plurality of such units may be used for treating an olefin stream. For example, when an oxygenate adsorption unit 20 becomes spent, e.g., its capacity for the adsorption of water and/or oxygenates is exhausted, the feed of olefin stream 15 thereto may be terminated. Thereafter, the spent oxygenate adsorption unit 20′ may be regenerated by a regenerant stream 35, as described hereinbelow, while an oxygenate adsorption unit 20, positioned in parallel, may be put online to receive olefin stream 15. In an embodiment, prior to the regeneration of a spent oxygenate adsorption unit 20′, residual olefins 48 may be recovered from spent oxygenate adsorption unit 20′ (see, for example, FIG. 2).

FIG. 2 schematically represents the treatment of a spent oxygenate adsorption unit 20′ for the removal of residual olefins 48 therefrom, according to another embodiment of the present invention. An oxygenate adsorption unit 20 that is spent may be designated herein as spent oxygenate adsorption unit 20′. As described with reference to FIG. 1, supra, when oxygenate adsorption unit 20 is spent, the feed of olefin stream 15 thereto may be terminated, and the spent oxygenate adsorption unit 20′ may be taken offline for regeneration. For example, in one embodiment, the process further comprises: when the oxygenate adsorption unit 20 is spent, terminating the feeding of an olefin stream 15 to the oxygenate adsorption unit 20; and prior to desorbing the oxygenates from the oxygenate adsorption unit 20, recovering the residual olefins 48 from a spent oxygenate adsorption unit 20′.

With further reference to FIG. 2, prior to the regeneration of spent oxygenate adsorption unit 20′, residual olefins 48 may be recovered therefrom by feeding a flushing stream 44 to spent oxygenate adsorption unit 20′ via line 46. Flushing stream 44 may comprise a dry hydrocarbon stream, e.g., comprising isobutane. Flushing stream 44 may have a temperature typically not more than 150° F. (65.56 degree Celsius), or in the range from 50° F. (10 degree Celsius) to 150° F. (65.56 degree Celsius). In an embodiment, residual olefins 48 may be combined, via line 52, with olefin stream 15. Following the recovery of residual olefins 48, spent oxygenate adsorption unit 20′ may be regenerated, e.g., as described hereinbelow. In an embodiment, a step of recovering the residual olefins 48 from spent oxygenate adsorption unit 20′may be omitted.

With further reference to FIG. 1, for the regeneration of spent oxygenate adsorption unit 20′, a regenerant stream 35 may be fed via line 28 to a first heating unit 30 such that regenerant stream 35 may attain a temperature of at least 250° F. (121.1 degree Celsius), and typically the regenerant stream 35 may attain a temperature in the range from 350 to 600° F. (176.7 to 315.6 degree Celsius). In an embodiment, first heating unit 30 may comprise a heat exchanger.

A regenerant stream 35 that is heated may be fed via line 32 to spent oxygenate adsorption unit 20′. In an embodiment, the feed of the regenerant stream 35 that is heated to the spent oxygenate adsorption unit 20′ (regeneration mode) may be in a direction opposite to that of olefin stream 15 to oxygenate adsorption unit 20 (adsorption mode). In an embodiment, regenerant stream 35 may comprise hydrocarbon vapor, e.g., comprising isobutane.

Water and oxygenates may be desorbed from the spent oxygenate adsorption unit 20′ by regenerant stream 35 to provide an oxygenated regenerant stream 45 comprising the water and oxygenates. Oxygenated regenerant stream 45 may be subjected to hydro-deoxygenation in hydro-deoxygenation zone 50 for the conversion of the oxygenates into paraffins and water. In an embodiment, regenerant stream 35 may be at a temperature below that suitable for the hydro-deoxygenation reaction. For example, as regeneration commences the spent oxygenate adsorption unit 20′ may initially serve to cool the regenerant stream 35.

Accordingly, oxygenated regenerant stream 45 may be fed via line 34 to a second heating unit 40 for heating the oxygenated regenerant stream 45. In an embodiment, second heating unit 40 may be used for heating the oxygenated regenerant stream 45 to a temperature in the range from 350 to 650° F. (176.7 to 343.3 degree Celsius), or from 400 to 500° F. (204.4 to 260 degree Celsius). As the system heats up, the duty of second heating unit 40 may be reduced to maintain the temperature of the inlet to hydro-deoxygenation zone 50. In an embodiment, second heating unit 40 may comprise a heat exchanger.

The oxygenated regenerant stream 45 that is heated may be sent via line 36 towards hydro-deoxygenation zone 50. Hydrogen gas may be injected via line 38 into the oxygenated regenerant stream 45 that is heated. In one embodiment, the injecting of the hydrogen gas into the oxygenated regenerant stream 45 is done at a location upstream from the hydro-deoxygenation zone 50. In an embodiment, the injection of hydrogen gas into the oxygenated regenerant stream 45 that is heated may be performed at a location upstream from hydro-deoxygenation zone 50. In an embodiment, a hydrogen to oxygenated regenerant stream feed ratio may be in the range from 50 to 750 standard cubic feet per barrel (SCF/bbl), or from 50 to 500 SCF/bbl. The oxygenated regenerant stream 45 and hydrogen gas may be contacted with a hydro-deoxygenation catalyst in hydro-deoxygenation zone 50 under hydro-deoxygenation conditions, such that oxygenates in oxygenated regenerant stream 45 may be converted to paraffins and water. The feed of oxygenated regenerant stream 45 to hydro-deoxygenation zone 50 may be upflow or downflow.

The hydro-deoxygenation zone effluent may be fed via line 54 to a cooling unit 60, such that at least a portion of the water of hydro-deoxygenation zone effluent may be separated as condensate. The condensed free water may be permanently removed, e.g., via line 57, to a waste water treatment unit (not shown). The residual effluent may be fed via line 58 to a gravity settler 70 for the separation of residual water, a liquid hydrocarbon phase 64, and hydrogen gas. In an embodiment, gravity settler 70 may comprise a three phase separator and/or a coalescer.

The residual water from gravity settler 70 may be permanently removed from gravity settler 70 via line 62 to the waste water treatment unit. The free water separated from the residual effluent via gravity settler 70 may be referred to herein as “residual water” so as to distinguish it from “condensed water” that was removed from the hydro-deoxygenation effluent by condensation upstream from gravity settler 70, it being understood that at least a portion of the residual water may be subsequently condensed from the residual effluent.

The liquid hydrocarbon phase 64 from gravity settler 70 may comprise oxygenate-derived paraffins as well as hydrocarbon components (e.g., isobutane) from the regenerant stream 35. Liquid hydrocarbon phase 64 may be used for various unit operations. The liquid hydrocarbon phase 64 may comprise a relatively small amount of dissolved water. In an embodiment, liquid hydrocarbon phase 64 may be sent to one or more dryers. In an embodiment, liquid hydrocarbon phase 64 may be combined with olefin stream 15 for drying via oxygenate adsorption unit 20. The hydrogen gas from gravity settler 70 may be sent, for example, to a refinery fuel gas header (not shown) for combustion.

In an embodiment, there is provided herein a process for eliminating oxygenates from a light hydrocarbon processing system. Such process may comprise feeding an olefin stream 15 to an oxygenate adsorption unit 20 to provide a deoxygenated olefin stream 25. In an embodiment, deoxygenated olefin stream 25 provided by oxygenate adsorption unit 20 may have an oxygenate content of not more than 5 ppmw, not more than 2 ppmw, or not more than 1 ppmw. In an embodiment, deoxygenated olefin stream 25 may have a water content of not more than 5 ppmw, not more than 2 ppmw, or not more than 1 ppmw. In an embodiment, the deoxygenated olefin stream 25 and an isoparaffin stream 102 may be contacted with an ionic liquid catalyst 108 in an ionic liquid alkylation zone 120 under ionic liquid alkylation conditions to provide an ionic liquid alkylate (see, for example, FIG. 3).

As a result of the feeding step, oxygenates and/or water may be adsorbed from the olefin stream 15 by oxygenate adsorption unit 20, and eventually the oxygenate adsorption unit 20 may become spent. When the oxygenate adsorption unit is spent, the step of feeding the olefin stream 15 thereto may be terminated. Such termination of the feeding step may signal the conclusion of the adsorption mode, and the oxygenate adsorption unit 20/20′ may then transition, or alternate, to the regeneration mode, during which oxygenates may be desorbed from the spent oxygenate adsorption unit 20′. In an embodiment, residual olefins 48 may be recovered from the spent oxygenate adsorption unit 20′ prior to the step of desorbing the oxygenates therefrom.

After the feeding step, and after any recovery of residual olefins 48 from the spent oxygenate adsorption unit 20′, oxygenates may be desorbed from the spent oxygenate adsorption unit 20′ via a regenerant stream 35 to provide an oxygenated regenerant stream 45 comprising the oxygenates. The step of desorbing oxygenates from the spent oxygenate adsorption unit 20′ may comprise heating the regenerant stream 35 to a temperature of at least 250° F. (121.1 degree Celsius), or to a temperature in the range from 350 to 600° F. (176.7 to 315.6 degree Celsius). Thereafter, the regenerant stream 35 that is heated may be passed through the spent oxygenate adsorption unit 20′. For example, in one embodiment, the desorbing of the oxygenates from the oxygenate adsorption unit 20 comprises heating the regenerant stream 35 to a temperature of at least 250° F. (121.1 degree Celsius), and thereafter passing the regenerant stream 35 through the oxygenate adsorption unit 20. In an embodiment, the regenerant stream 35 may comprise a hydrocarbon (e.g., isobutane) vapor.

After the desorbing step, the oxygenates of the oxygenated regenerant stream 45 may be converted to paraffins and water. The step of converting the oxygenates of the oxygenated regenerant stream to paraffins and water may comprise contacting the oxygenated regenerant stream 45 with a hydro-deoxygenation catalyst in the presence of hydrogen gas in a hydro-deoxygenation zone 50 under hydro-deoxygenation conditions. In an embodiment, the hydro-deoxygenation catalyst may comprise a noble metal on a suitable support. In an embodiment, the hydro-deoxygenation catalyst may comprise a noble metal selected from the group consisting of Pt, Pd, and combinations thereof.

Prior to the step of contacting the oxygenated regenerant stream 45 with a hydro-deoxygenation catalyst, the oxygenated regenerant stream may be heated to a suitable hydro-deoxygenation temperature. In an embodiment, the oxygenated regenerant stream 45 may be heated to a temperature in the range from 350 to 650° F. (176.7 to 343.3 degree Celsius), or from 400 to 500° F. (204.4 to 260 degree Celsius).

After the step of heating the oxygenated regenerant stream 45 to a suitable hydro-deoxygenation temperature, hydrogen gas may be injected into the oxygenated regenerant stream. In an embodiment, the hydrogen gas may be injected into the oxygenated regenerant stream 45 at a location upstream from hydro-deoxygenation zone 50.

In an embodiment, the hydro-deoxygenation conditions may comprise a temperature in the range from 350 to 650° F. (176.7 to 343.3 degree Celsius), or from 400 to 500° F. (204.4 to 260 degree Celsius). The hydro-deoxygenation conditions may further comprise a pressure in the range from 100 to 400 psig, or from 100 to 300 psig. The hydro-deoxygenation conditions may still further comprise a liquid hourly space velocity (LHSV) in the range from 2 to 20 hr⁻¹, or from 2 to 10 hr⁻¹.

After the step of contacting the oxygenated regenerant stream 45 with a hydro-deoxygenation catalyst, the hydro-deoxygenation zone effluent may be cooled to condense at least a portion of the water from the hydro-deoxygenation zone effluent to provide condensed water and a residual effluent. The residual effluent may comprise hydrogen gas and residual water, as well as oxygenate-derived paraffins and hydrocarbon components of the regenerant. The hydrogen gas and residual water may be separated from the residual effluent. Both the condensed water and the residual water may be permanently removed from the system.

In another embodiment, there is provided herein a process for eliminating oxygenates from a light hydrocarbon processing system. Such process may comprise removing oxygenates from an olefin stream 15 via an oxygenate adsorption unit 20 to provide a deoxygenated olefin stream 25, wherein the oxygenate adsorption unit becomes spent. In an embodiment, olefin stream 15 may comprise light hydrocarbons, e.g., C₃-C₅ olefins.

An olefin stream 15 that is fed to oxygenate adsorption unit 20 may be raw or untreated. In an embodiment, olefin stream 15 may be from a FCC unit (not shown). Olefin stream 15 may be contaminated with both water and various oxygenates. Olefin stream 15 may be saturated with water vapor. In an embodiment, olefin stream 15 may have a water content of at least 300 ppmw, or in the range from 300 to 500 ppmw.

The deoxygenated olefin stream 25 provided by oxygenate adsorption unit 20 may have an oxygenate content of not more than 5 ppmw, not more than 2 ppmw, or not more than 1 ppmw. In an embodiment, deoxygenated olefin stream 25 may have a water content of not more than 5 ppmw, not more than 2 ppmw, or not more than 1 ppmw. In an embodiment, deoxygenated olefin stream 25 and an isoparaffin stream 102 may be contacted with an ionic liquid catalyst 108 in an ionic liquid alkylation zone 120 under ionic liquid alkylation conditions to provide an ionic liquid alkylate (see, for example, FIG. 3).

As a result of the step of removing oxygenates from olefin stream 15, oxygenate adsorption unit 20 may become spent. Prior to the regeneration of the spent oxygenate adsorption unit 20′, residual olefins 48 may be flushed therefrom for recovery. In an embodiment, the residual olefins 48 may be flushed from the spent oxygenate adsorption unit 20′ via an isobutane stream. In an embodiment, the isobutane stream for the recovery of the residual olefins 48 may have a temperature of not more than 150° F. (65.56 degree Celsius), or from 50 to 150° F. (10 to 65.56 degree Celsius). The residual (flushed) olefins can be combined with olefin stream 15, or may be fed to a FCC Gas Recovery Unit (not shown).

A spent oxygenate adsorption unit 20′may be regenerated via a regenerant stream 35 to provide an oxygenated regenerant stream 45 comprising the oxygenates, wherein the oxygenates of the oxygenated regenerant stream may be desorbed from spent oxygenate adsorption unit 20′ by the regenerant stream 35. In an embodiment, the regenerant stream 35 may have a temperature of at least 250° F. (121.1 degree Celsius), or from 300 to 600° F. (148.9 to 315.6 degree Celsius). The oxygenated regenerant stream may be contacted with a hydro-deoxygenation catalyst, in the presence of hydrogen gas in a hydro-deoxygenation zone 50 under hydro-deoxygenation conditions, to convert the oxygenates of the oxygenated regenerant stream to paraffins and water.

Typical hydro-deoxygenation conditions may comprise a temperature in the range from 350 to 650° F. (176.7 to 343.3 degree Celsius), or from 400 to 500° F. (204.4 to 260 degree Celsius); and a pressure in the range from 100 to 400 psig, or from 100 to 300 psig. The hydro-deoxygenation conditions may still further comprise an LHSV in the range from 2 to 20 hr⁻¹, or from 2 to 10 hr⁻¹. In an embodiment, the hydro-deoxygenation catalyst may comprise a noble metal selected from the group consisting of Pt, Pd, and combinations thereof.

The effluent from hydro-deoxygenation zone 50 may be referred to herein as a hydro-deoxygenation zone effluent. The hydro-deoxygenation zone effluent may be cooled to condense at least a portion of the water from the hydro-deoxygenation zone effluent to provide condensed water and a residual effluent comprising residual water. The condensed water may be permanently removed from the system, for example, by sending the condensed water to a waste water treatment unit. The residual effluent may be fed to a gravity settler 70. In an embodiment, the gravity settler 70 may comprise a coalescer.

The residual effluent may comprise the residual water, liquid hydrocarbons, and hydrogen gas. Via the gravity settler 70, the residual water, a liquid hydrocarbon phase, and hydrogen gas may each be separated from the residual effluent (see, for example, FIG. 1). The residual water may be permanently removed from the system, for example, by sending the residual water to the waste water treatment unit. The liquid hydrocarbon phase 64 may comprise oxygenate-derived paraffins as well as hydrocarbon components (e.g., isobutane) of the regenerant stream 35. The hydrogen gas separated from the hydro-deoxygenation zone effluent may be sent to a refinery fuel gas header.

FIG. 3 schematically represents a system and process for ionic liquid catalyzed alkylation, according to another embodiment of the present invention. Such system and process may use a dry, deoxygenated olefin stream as a feed for the ionic liquid alkylation reaction. Ionic liquid alkylation system 100 (see, for example, FIG. 3) provides a non-limiting example of a light hydrocarbon processing system to which oxygenate removal processes of the present invention may be applied.

A process for the preparation of ionic liquid alkylate will now be described with reference to FIG. 3. An olefin stream 15 may be fed via line 18 to an oxygenate adsorption unit 20 to provide a dewatered and deoxygenated olefin stream 25, e.g., essentially as described with reference to FIG. 1, supra. At the same time, an isoparaffin stream 102 may be fed via line 104 to an isoparaffin dryer 110 to provide a dried isoparaffin stream. The deoxygenated olefin stream 25 and the dried isoparaffin stream may be fed, via lines 22 and 106, respectively, to an ionic liquid alkylation zone 120 together with an ionic liquid catalyst 108.

In ionic liquid alkylation zone 120, at least one isoparaffin and at least one olefin may be contacted with ionic liquid catalyst 108 under ionic liquid alkylation conditions. Anhydrous HCl co-catalyst or an organic chloride catalyst promoter (neither of which are shown) may be combined with the ionic liquid in ionic liquid alkylation zone 120 to attain the desired level of catalytic activity and selectivity for the alkylation reaction. Ionic liquid alkylation conditions, feedstocks, and ionic liquid catalysts that may be suitable for performing ionic liquid alkylation reactions in ionic liquid alkylation system 100 are described, for example, hereinbelow.

The effluent from ionic liquid alkylation zone 120 may be fed via line 122 to an ionic liquid/hydrocarbon (IL/HC) separator 130 for the separation of a hydrocarbon phase from the effluent. Non-limiting examples of separation processes that can be used for separating the hydrocarbon phase from the effluent include coalescence, phase separation, extraction, membrane separation, and partial condensation. IL/HC separator 130 may comprise, for example, one or more of the following: a settler, a coalescer, a centrifuge, a distillation column, a condenser, and a filter.

The hydrocarbon phase from IL/HC separator 130 may be fed via line 132 to an ionic liquid alkylate separation system 140. The hydrocarbon phase from IL/HC separator 130 may be referred to herein as an alkylation hydrocarbon phase. Ionic liquid alkylate separation system 140 may comprise at least one distillation unit (not shown). The alkylation hydrocarbon phase from IL/HC separator 130 may be fractionated via ionic liquid alkylate separation system 140 to provide an alkylate product 144, as well as HCl 146, a propane fraction 148, an n-butane fraction 150, and an isobutane fraction 152.

The instant specification further provides a process for eliminating oxygenates from a hydrocarbon processing system. With further reference to FIGS. 1 and 3, oxygenates may be effectively removed from an olefin stream 15 by feeding the olefin stream 15 to oxygenate adsorption unit 20 in the adsorption mode to provide a deoxygenated olefin stream 25. Oxygenate adsorption unit 20 may also remove water from olefin stream 15 concomitantly with the removal of oxygenates. In an embodiment, deoxygenated olefin stream 25 may have a water content of not more than 5 ppmw, not more than 2 ppmw, or not more than 1 ppmw. In an embodiment, deoxygenated olefin stream 25 may have an oxygenate content of not more than 5 ppmw, not more than 2 ppmw, or not more than 1 ppmw.

With further reference to FIG. 3, the deoxygenated olefin stream 25 and an isoparaffin stream 102 may be contacted with an ionic liquid catalyst 108 in an ionic liquid alkylation zone 120 under ionic liquid alkylation conditions. An alkylation hydrocarbon phase may be separated from an effluent of ionic liquid alkylation zone 120, e.g., using an IL/HC separator 130. Thereafter, the alkylation hydrocarbon phase may be fractionated, e.g., via an ionic liquid alkylate separation system 140, to provide, inter alia, an alkylate product 144.

With still further reference to FIG. 1, when an oxygenate adsorption unit 20 becomes spent, the feed of olefin stream 15 to the spent oxygenate adsorption unit 20′ may be terminated, preparatory to operation of the spent oxygenate adsorption unit 20′ in the regeneration mode. Spent oxygenate adsorption unit 20′ may be regenerated via a regenerant stream 35 to provide an oxygenated regenerant stream 45 comprising desorbed oxygenates. Oxygenated regenerant stream 45 may further comprise desorbed water. The oxygenates of oxygenated regenerant stream 45 may be eliminated from the system by converting the oxygenates to paraffins and water.

In an embodiment, the conversion of the oxygenates in oxygenated regenerant stream 45 to paraffins and water may involve heating the oxygenated regenerant stream to a temperature in the range from 350 to 650° F. (176.7 to 343.3 degree Celsius). Thereafter, hydrogen gas may be injected into the oxygenated regenerant stream at a location upstream from a hydro-deoxygenation zone 50. Thereafter, the oxygenated regenerant stream and hydrogen gas may be contacted with a hydro-deoxygenation catalyst in hydro-deoxygenation zone 50 under hydro-deoxygenation conditions. In an embodiment, the hydrogen gas may be injected at a rate in the range from 50 to 500 standard cubic feet per barrel (SCF/bbl) of the oxygenated regenerant stream 45. Typical hydro-deoxygenation conditions may comprise a temperature in the range from 350 to 650° F. (176.7 to 343.3 degree Celsius), a pressure in the range from 100 to 400 psig, and an LHSV in the range from 2 to 20 hr⁻¹.

Ionic Liquid Catalyzed Alkylation

Ionic liquid catalysts may be useful for a range of hydrocarbon conversion reactions, including alkylation reactions for the production of alkylate, e.g., comprising gasoline blending components, and the like. In an embodiment, feedstocks for ionic liquid catalyzed alkylation may comprise various olefin- and isoparaffin containing hydrocarbon streams in or from one or more of the following: a petroleum refinery, a gas-to-liquid conversion plant, a coal-to-liquid conversion plant, a naphtha cracker, a middle distillate cracker, and a wax cracker, and the like.

Examples of olefin containing streams include FCC off-gas, coker gas, olefin metathesis unit off-gas, polyolefin gasoline unit off-gas, methanol to olefin unit off-gas, FCC light naphtha, coker light naphtha, Fischer-Tropsch unit condensate, and cracked naphtha. Some olefin containing streams may contain two or more olefins selected from ethylene, propylene, butylenes, pentenes, and up to C₁₀ olefins. Such olefin containing streams are further described, for example, in U.S. Pat. No. 7,572,943, the disclosure of which is incorporated by reference herein in its entirety.

Examples of isoparaffin containing streams include, but are not limited to, FCC naphtha, hydrocracker naphtha, coker naphtha, Fisher-Tropsch unit condensate, and cracked naphtha. Such streams may comprise at least one C₄-C₁₀ isoparaffin. In an embodiment, such streams may comprise a mixture of two or more isoparaffins. In a sub-embodiment, an isoparaffin feed to the alkylation reactor during an ionic liquid catalyzed alkylation process may comprise isobutane.

Various ionic liquids may be used as catalysts for alkylation reactions involving olefins. Ionic liquids are generally organic salts with melting points below 100° C. (212 degree Fahrenheit) and often below room temperature. The use of chloroaluminate ionic liquids as alkylation catalysts in petroleum refining has been described, for example, in commonly assigned U.S. Pat. Nos. 7,531,707, 7,569,740, and 7,732,654, the disclosure of each of which is incorporated by reference herein in its entirety. Exemplary ionic liquids for use as catalysts in ionic liquid catalyzed alkylation reactions may comprise at least one compound of the general formulas A and B:

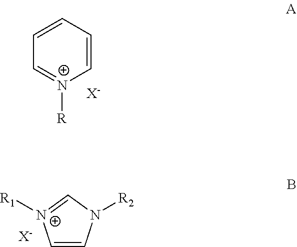

wherein R is H, methyl, ethyl, propyl, butyl, pentyl or hexyl, each of R₁ and R₂ is H, methyl, ethyl, propyl, butyl, pentyl or hexyl, wherein R₁ and R₂ may or may not be the same, and X is a chloroaluminate.

Non-limiting examples of chloroaluminate ionic liquid catalysts that may be used in alkylation processes according to embodiments of the instant invention include those comprising 1-butyl-4-methyl-pyridinium chloroaluminate, 1-butyl-3-methyl-imidazolium chloroaluminate, 1-H-pyridinium chloroaluminate, N-butylpyridinium chloroaluminate, and mixtures thereof.

Exemplary reaction conditions for ionic liquid catalyzed alkylation are as follows. The ionic liquid alkylation reaction temperature may be generally in the range from −40° C. to +250° C. (−40° F. to +482° F.), typically from −20° C. to +100° C. (−4° F. to +212° F.), and often from +4° C. to +60° C. (+39.2° F. to +140° F.). The ionic liquid alkylation reactor pressure may be in the range from atmospheric pressure to 8000 kPa. Typically, the pressure in the ionic liquid alkylation zone 120 is sufficient to keep the reactants in the liquid phase.

Residence time of reactants in ionic liquid alkylation zone 120 may generally be in the range from a few seconds to hours, and usually from 0.5 min to 60 min. A feed stream introduced into ionic liquid alkylation zone 120 may have an isoparaffin:olefin molar ratio generally in the range from 1 to 100, more typically from 2 to 50, and often from 2 to 20.

The volume of ionic liquid catalyst 108 in ionic liquid alkylation zone 120 may be generally in the range from 1 to 70 vol %, and usually from 4 to 50 vol %. The ionic liquid alkylation conditions may be adjusted to optimize process performance for a particular process or targeted product(s).

Numerous variations on the present invention are possible in light of the teachings described herein. It is therefore understood that within the scope of the following claims, the invention may be practiced otherwise than as specifically described or exemplified herein.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance.

All of the publications, patents and patent applications cited in this application are herein incorporated by reference in their entirety to the same extent as if the disclosure of each individual publication, patent application or patent was specifically and individually indicated to be incorporated by reference in its entirety.

This written description uses examples to disclose the invention, including the best mode, and also to enable any person skilled in the art to make and use the invention. Many modifications of the exemplary embodiments of the invention disclosed above will readily occur to those skilled in the art. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

The invention illustratively disclosed herein suitably may be practiced in the absence of any element which is not specifically disclosed herein.

It is claimed:

1. A process for eliminating oxygenates from a light hydrocarbon processing system, the process comprising:
a) feeding an olefin stream to an oxygenate adsorption unit to provide a deoxygenated olefin stream;
b) after step a), desorbing the oxygenates from the oxygenate adsorption unit via a regenerant stream to provide an oxygenated regenerant stream comprising the oxygenates; and
c) converting the oxygenates of the oxygenated regenerant stream to oxygenate-derived paraffins and water.

2. The process of claim 1, wherein step c) comprises:
d) contacting the oxygenated regenerant stream with a hydro-deoxygenation catalyst in a presence of a hydrogen gas in a hydro-deoxygenation zone under hydro-deoxygenation conditions.

3. The process of claim 2, further comprising:
e) prior to step d), heating the oxygenated regenerant stream to a temperature from 350 to 650° F. (176.7 to 343.3 degree Celsius).

4. The process of claim 3, further comprising:
f) after step e), injecting the hydrogen gas into the oxygenated regenerant stream at a location upstream from the hydro-deoxygenation zone.

5. The process of claim 2, wherein the hydro-deoxygenation conditions comprise a temperature from 350 to 650° F. (176.7 to 343.3 degree Celsius), a pressure from 100 to 400 psig, and an LHSV from 2 to 20 hr⁻¹.

6. The process of claim 2, further comprising:
g) cooling a hydro-deoxygenation zone effluent to condense at least a portion of the water from the hydro-deoxygenation zone effluent to provide condensed water and a residual effluent;
h) separating the hydrogen gas and residual water from the residual effluent; and
i) permanently removing the condensed water and the residual water from the light hydrocarbon processing system.

7. The process of claim 1, further comprising:
j) when the oxygenate adsorption unit is spent, terminating step a); and
k) prior to step b), recovering residual olefins from a spent oxygenate adsorption unit.

8. The process of claim 1, wherein step b) comprises heating the regenerant stream to a temperature of at least 250° F. (121.1 degree Celsius), and thereafter passing the regenerant stream through the oxygenate adsorption unit.

9. The process of claim 1, wherein step a) comprises adsorbing water and the oxygenates from the olefin stream via the oxygenate adsorption unit.

10. The process of claim 1, wherein the deoxygenated olefin stream provided by the oxygenate adsorption unit has an oxygenate content of not more than 5 ppmw and a water content of not more than 5 ppmw.

11. The process of claim 1, further comprising:
l) contacting the deoxygenated olefin stream and an isoparaffin stream with an ionic liquid catalyst in an ionic liquid alkylation zone under ionic liquid alkylation conditions to provide an ionic liquid alkylate.

12. A process for eliminating oxygenates from a light hydrocarbon processing system, the process comprising:
a) removing the oxygenates from an olefin stream via an oxygenate adsorption unit to provide a deoxygenated olefin stream, wherein the oxygenate adsorption unit becomes spent;
b) regenerating a spent oxygenate adsorption unit via a regenerant stream to provide an oxygenated regenerant stream comprising the oxygenates; and
c) contacting the oxygenated regenerant stream with a hydro-deoxygenation catalyst in a presence of a hydrogen gas in a hydro-deoxygenation zone under hydro-deoxygenation conditions, wherein the oxygenates of the oxygenated regenerant stream are converted to oxygenate-derived paraffins and water.

13. The process of claim 12, wherein:
the hydro-deoxygenation conditions comprise a temperature from 350 to 650° F. (176.7 to 343.3 degree Celsius), a pressure from 100 to 400 psig, and an LHSV from 2 to 20 hr⁻¹, and
the hydro-deoxygenation catalyst comprises a noble metal selected from the group consisting of Pt, Pd, and combinations thereof.

14. The process of claim 12, further comprising:
d) cooling a hydro-deoxygenation zone effluent to condense at least a portion of the water from the hydro-deoxygenation zone effluent to provide condensed water and a residual effluent;
e) separating a residual water, via a gravity settler, from the residual effluent; and
f) permanently removing the condensed water and the residual water from the light hydrocarbon processing system.

15. The process of claim 12, further comprising:
g) prior to step b), flushing residual olefins from the spent oxygenate adsorption unit with a flushing stream having a temperature of not more than 150° F. (65.56 degree Celsius).

16. The process of claim 12, wherein the regenerant stream has a temperature of at least 250° F. (121.1 degree Celsius).

17. The process of claim 12, wherein:
the deoxygenated olefin stream provided by the oxygenate adsorption unit has an oxygenate content of not more than 5 ppmw, and the process further comprises:
h) contacting the deoxygenated olefin stream and an isoparaffin stream with an ionic liquid catalyst in an ionic liquid alkylation zone under ionic liquid alkylation conditions to provide an ionic liquid alkylate.

18. A process for eliminating oxygenates from a light hydrocarbon processing system, the process comprising:
a) feeding an olefin stream to an oxygenate adsorption unit to provide a deoxygenated olefin stream;
b) contacting the deoxygenated olefin stream and an isoparaffin stream with an ionic liquid catalyst in an ionic liquid alkylation zone under ionic liquid alkylation conditions;
c) separating an alkylation hydrocarbon phase from an effluent of the ionic liquid alkylation zone;
d) fractionating the alkylation hydrocarbon phase to provide an alkylate product;
e) when the oxygenate adsorption unit becomes spent, regenerating a spent oxygenate adsorption unit via a regenerant stream to provide an oxygenated regenerant stream comprising the oxygenates; and
f) converting the oxygenates of the oxygenated regenerant stream to oxygenate-derived paraffins and water.

19. The process of claim 18, wherein step f) comprises:
g) heating the oxygenated regenerant stream to a temperature from 350 to 650° F. (176.7 to 343.3 degree Celsius);
h) after step g), injecting a hydrogen gas into the oxygenated regenerant stream at a location upstream from a hydro-deoxygenation zone; and
i) contacting the oxygenated regenerant stream and the hydrogen gas with a hydro-deoxygenation catalyst in the hydro-deoxygenation zone under hydro-deoxygenation conditions.

20. The process of claim 19, wherein:
the hydro-deoxygenation conditions comprise the temperature from 350 to 650° F. (176.7 to 343.3 degree Celsius), a pressure from 100 to 400 psig, and an LHSV from 2 to 20 hr⁻¹, and
step h) comprises injecting the hydrogen gas at a rate from 50 to 500 standard cubic feet per barrel of the oxygenated regenerant stream.

21. The process of claim 1, additionally comprising:
removing the water from the oxygenate-derived paraffins to make a liquid hydrocarbon phase and combining the liquid hydrocarbon phase with the olefin stream that is fed to the oxygenate adsorption unit in step a).

22. The process of claim 12, additionally comprising:
removing the water from the oxygenate-derived paraffins to make a liquid hydrocarbon phase and combining the liquid hydrocarbon phase with the olefin stream in step a).

23. The process of claim 18, additionally comprising:
removing the water from the oxygenate-derived paraffins to make a liquid hydrocarbon phase and combining the liquid hydrocarbon phase with the olefin stream that is fed to the oxygenate adsorption unit in step a).
